(19) United States
(12) Patent Application Publication
(10) Pub. No.: US 2004/0228893 A1
(43) Pub. Date: Nov. 18, 2004

(54) Increasing the stability of cosmetic formulations by addition of iminodisuccinic acid

(75) Inventors: Rainer Kropke, (US); Jens Nielsen, (US); Anja Goppel, (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/791,354

(22) Filed: Mar. 1, 2004

Related U.S. Application Data
(63) Continuation of application No. PCT/EP02/09576, filed on Aug. 28, 2002.

(30) Foreign Application Priority Data
Sep. 1, 2001 (DE) 10142927.4

(51) Int. Cl. A61K 7/42; A61K 7/021

(52) U.S. Cl. 424/401; 424/63

(57) ABSTRACT

The present invention is a method for increasing at least one of the color-stability and the light-stability of a cosmetic or dermatological formulation, comprising the step of adding iminodisuccinic acid or a salt thereof to the cosmetic or dermatological formulation. The invention also includes a cosmetic or dermatological formulation comprising iminodisuccinic acid or a salt thereof and a cosmetic or dermatological product comprising the cosmetic or dermatological formulation and transparent or translucent packaging.

### CROSS-REFERENCE TO RELATED APPLICATIONS

This is a continuation application of PCT/EP02/09576, filed Aug. 28, 2002, which is incorporated herein by reference in its entirety, and also claims the benefit of German Priority Application No. 101 42 927.4, filed Sep. 1, 2001.

### FIELD OF THE INVENTION

The present invention relates to cosmetic or dermatological formulations, the stability of which is increased decisively by addition of iminodisuccinic acid or its salts, and to their use.

### BACKGROUND OF THE INVENTION

Cosmetic and dermatological formulations that are transparent or translucent are extremely popular with consumers. Since they are usually transparent, and are probably often colored, but just as often colorless and clear, they offer the cosmetic developer additional design possibilities which in part have a functional character but in part also serve merely to improve the external appearance. Thus, for example, interesting optical effects can be imparted to the product, which is then as a rule presented to the observer in transparent packaging, by incorporated colored pigments, gas bubbles and the like, or also larger objects.

When it is desirable for the product to incorporate objects, whether they may be detectable as such with the naked eye or they result in visible forms in microscopic dimensions but in an interesting arrangement, for example in the form of artificially produced streaks of color, it is desirable for these products to be stored in transparent or translucent packaging.

Transparent and translucent packaging is also always more popular for cosmetic and dermatological formulations which are not transparent or translucent. Transparent glass jars and bottles belong to the most widely used forms of packaging for cosmetic and dermatological formulations. In their often artistically decorated embodiments, they have a particularly fine effect and give the consumer the impression of having acquired with the cosmetic a small treasure and a high-quality product.

Unfortunately, however, cosmetic and dermatological formulations often are light- and color-stable to only a limited extent, and for this reason they must be stored protected from light, e.g., in plastic bottles.

### SUMMARY OF THE INVENTION

It was therefore the object of the present invention to stabilize cosmetic and dermatological formulations such that they can be stored and made available to consumers in transparent or translucent packaging without problems.

The object was surprisingly achieved by the use of iminodisuccinic acid or salts thereof to increase the color- and light-stability of cosmetic and dermatological formulations.

In this context, it is advantageous according to the invention to employ iminodisuccinic acid or salts thereof in a concentration of 0.001 to 15% by weight, based on the total weight of the formulation.

It is advantageous here according to the invention to employ the tetrasodium salt as the preferred iminodisuccinic acid compound.

### DETAILED DESCRIPTION OF THE PREFERRED EMBODIMENT

Iminodisuccinic acid has the following structure, an equilibrium of tautomeric forms supposedly being present:

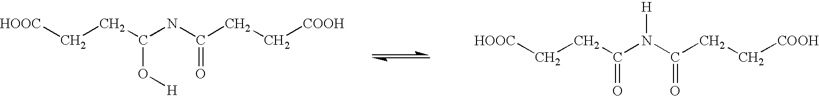

Iminodisuccinic acid is obtainable as a solid, inter alia, from Bayer AG under the trade name Iminodisuccinate VP OC 370 (approx. 30% strength solution) and Baypure CX 100.

The use of iminodisuccinic acid or salts thereof leads to an increase in the color-, light-and smell-stability of cosmetic and dermatological formulations. In particular, the color-, light- and smell-stability of cosmetic and dermatological formulations in transparent or translucent packaging is increased by the formulations according to the invention.

Products comprising a cosmetic and dermatological formulation comprising iminodisuccinic acid or salts thereof and a transparent or translucent packaging are advantageous according to the invention.

These cosmetic and dermatological products according to the invention can advantageously be used as skin care products, as face care products and as sunscreen products. In the context of the invention, “skin care products” are understood here as meaning, inter alia, skin creams, skin lotions, milks, ointments, oils, balsams and sera which are used for care of the skin. Face care products are used as a special form of skin care products for care of facial skin. They are used in particular to prevent developing or to reduce already existing wrinkles and folds. According to the invention, face care products also include decorative cosmetics, the main purpose of which is to change the color of skin and skin appendages (e.g. eyelashes, eyebrows). Sunscreen products in the context of the invention are to be understood as meaning all forms of formulations that include UV light protection filters. They furthermore include so-called “aftersun products.” These are intended to cool the skin after sunbathing and to improve its moisture retention capacity, the imparting of the cooling effect playing a central role. This cooling effect is as a rule achieved by large amounts of ethanol and water, which evaporates spontaneously when the formulation is spread on the skin. These preparations furthermore usually comprise moisturizing agents, such as glycerol or propylene glycol, and anti-inflammatory compounds, such as, for example, allantion, α-bisabolol, panthenol or aloe vera extract.

The following examples are intended to illustrate the present invention without limiting it. Unless stated otherwise, all the amounts data, contents and percentage contents are based on the weight and the total amount or on the total weight of the formulations.

| | Example number | | | | | | | |
|---|---|---|---|---|---|---|---|---|
| | 1 | 2 | 3 | 4 | 5 | 6 | 7 | 8 |
| Glyceryl stearate citrate | 2 | | | | | | | |
| Glyceryl stearate | | 5 | 2 | 3 | | | | |
| PEG-40 stearate | | | 1 | | | | | |
| PEG-100 stearate | | | | 1 | | | | |
| Triglycerol methylglucose | | | | | 3 | | | |
| distearate | | | | | | | | |
| Sorbitan stearate | | | | | 1 | | | |
| Polyethylene glycol (21) | | | | | | 2 | | |
| stearyl ether | | | | | | | | |
| Polyethylene glycol (2) | | | | | | 1 | | |
| stearyl ether (steareth-2) | | | | | | | | |
| Cetearyl glucoside | | | | | | | 2 | |
| Stearic acid | | | | | | | | 2.5 |
| Myristyl myristate | 1 | | 1 | | | | 1 | |
| Behenyl alcohol | | | | | 1 | | | 2 |
| Stearyl alcohol | 2 | 1 | | | | | 5 | |
| Cetearyl alcohol | | | 2 | | | 2 | | 1 |
| Cetyl alcohol | 1 | | | 1 | 1 | | | |
| Hydrogenated coconut | 2 | | | 1 | | | | 1 |
| fatty glycerides | | | | | | | | |
| Shea butter | | 2 | | | | | | |
| C12-15 Alkyl benzoate | | 3 | 3 | | 2 | 5 | 2 | |
| Butylene glycol | 1 | | | | | | | 2 |
| dicaprylate/dicaprate | | | | | | | | |
| Caprylic/capric | | 4 | | | 1 | | | |
| triglycerides | | | | | | | | |
| Hydrogenated polydecene | | | | | | | 1 | |
| Ethylhexyl coconut fatty | 3 | | | | | | | 2 |
| acid ester | | | | | | | | |
| Octyldodecanol | | | | | | 1 | | |
| Mineral oil | | 1 | | | | 1 | | |
| Vaseline | 4 | | | 2 | | | | |
| Octamethyltetrasiloxane | | 1 | 3 | 1 | 3 | 2 | | |
| Dimethylpolysiloxane | | | 1 | | | | | 1 |
| Dicaprylyl ether | 1 | 4 | | | | | | |
| Dicarprylyl carbonate | | | | | 2 | | | 4 |
| Polydecene | | | | 1 | | | 5 | |
| TiO₂ | | | 1 | 1 | | | 2 | 1 |
| Ethylhexyl | 3 | 2 | | 5 | 3 | | | 3 |
| methoxycinnamate | | | | | | | | |
| 2-Ethylhexyl 2-cyano-3- | | | 5 | | | | | |
| diphenylacrylate | | | | | | | | |
| Ethylhexyltriazone | | 2 | | | 2 | 3 | | |
| Butylmethoxydibenzoyl- | | 1 | | 1 | | | | |
| methane | | | | | | | | |
| Bis-Ethylhexyloxyphenol- | 1 | | | | 1 | | | 2 |
| methoxyphenyltriazines | | | | | | | | |
| Ubiquinone (Q10) | 0.05 | | 0.05 | | 0.02 | | | 0.1 |
| Retinyl palmitate | | | | | | | 0.1 | |
| Tocopheryl acetate | | 0.5 | | | | | | |
| α-Glucosylrutin | | | | | | 0.1 | | |
| Ascorbic acid | | | | 0.2 | | | | |
| Liponic acid | | | 0.1 | | | | | |
| Retinol | | | | | 0.1 | | | |
| Iminodisuccinic acid | | 0.2 | | | | | | |
| Tetrasodium | 0.1 | — | 0.3 | 0.1 | 0.1 | 0.2 | 0.5 | 0.25 |
| iminodisuccinate | | | | | | | | |
| Phenoxyethanol | 0.3 | | | 0.8 | 0.4 | 0.5 | | 0.3 |
| p-Hydroxybenzoic acid | 0.5 | 0.4 | 0.3 | 0.4 | | | 0.4 | 0.6 |
| alkyl ester | | | | | | | | |
| Hexamidine diisothionate | | | 0.04 | 0.05 | | 0.1 | | |
| Diazolidinylurea | 0.25 | | | | 0.2 | | 0.1 | |
| 1,3-Dimethylol-5,5- | | 0.2 | | | | | | |
| dimethyl-hydantoin | | | | | | | | |
| Iodopropynyl butyl | 0.1 | | 0.05 | | | 0.20 | | |
| carbamate | | | | | | | | |
| Ethanol, denatured | 1 | 2 | | | 8 | | | 3 |
| 2-Ethylhexyl glycerol ether | | | | 3 | | | | |
| Xanthan gum | 0.1 | | | 0.2 | 0.1 | 0.1 | | |
| Polyacrylic acid | 0.2 | | 0.1 | | | 0.2 | | |
| Polyacrylamide | | 0.2 | | | 0.2 | | | |
| Glycerol | 8 | 10 | 5 | 15 | 5 | 6 | 4 | 5 |
| Butylene glycol | | 1 | 2 | | | | 2 | |
| Water- and/or oil-soluble | 0.05 | | | | | | | 0.1 |
| dyestuffs | | | | | | | | |
| Fillers (distarch | | | | | | | 5 | 1 |
| phosphate, SiO₂, talc, | | | | | | | | |
| aluminum stearate) | | | | | | | | |
| Perfume | q.s. | q.s. | q.s. | q.s. | q.s. | q.s. | q.s. | q.s. |
| Water | to 100 | to 100 | to 100 | to 100 | to 100 | to 100 | to 100 | to 100 |

That which is claimed:

1. A method for increasing at least one of the color-stability and the light-stability of a cosmetic or dermatological formulation, comprising the step of adding iminodisuccinic acid or a salt thereof to the cosmetic or dermatological formulation in an amount sufficient to increase at least one of the color-stability and the light-stability of a cosmetic or dermatological formulation.

2. The method according to claim 1, wherein said adding step includes adding iminodisuccinic acid.

3. The method according to claim 1, wherein said adding step includes adding the tetrasodium salt of iminodisuccinic acid.

4. The method according to claim 1, wherein the iminodisuccinic acid or a salt thereof is added in an amount from 0.001 to 15% by weight, based on the total weight of the formulation.

5. The method according to claim 1, wherein the iminodisuccinic acid or a salt thereof is added in an amount from 0.1 to 0.5% by weight, based on the total weight of the formulation.

6. The method according to claim 1, wherein said cosmetic or dermatological formulation is provided in transparent or translucent packaging.

7. The method according to claim 1, wherein said cosmetic or dermatological formulation is a skin care product.

8. The method according to claim 1, wherein said cosmetic or dermatological formulation is a face care product.

9. The method according to claim 1, wherein said cosmetic or dermatological formulation is a sunscreen product.

10. A cosmetic or dermatological formulation comprising iminodisuccinic acid or a salt thereof in an amount sufficient to increase at least one of the color-stability and the light-stability of a cosmetic or dermatological formulation.

11. The formulation according to claim 10, wherein said iminodisuccinic acid or a salt thereof includes iminodisuccinic acid.

12. The formulation according to claim 10, wherein said iminodisuccinic acid or a salt thereof includes the tetrasodium salt of iminodisuccinic acid.

13. The formulation according to claim 10, wherein said iminodisuccinic acid or a salt thereof is present in an amount from 0.001 to 15% by weight, based on the total weight of the formulation.

14. The formulation according to claim 10, wherein said iminodisuccinic acid or a salt thereof is present in an amount from 0.1 to 0.5% by weight, based on the total weight of the formulation.

15. A cosmetic or dermatological product comprising:
a cosmetic or dermatological formulation comprising iminodisuccinic acid or a salt thereof in an amount sufficient to increase at least one of the color-stability and the light-stability of a cosmetic or dermatological formulation; and
transparent or translucent packaging.

16. The product according to claim 15, wherein said cosmetic or dermatological formulation includes iminodisuccinic acid.

17. The product according to claim 15, wherein said cosmetic or dermatological formulation includes the tetrasodium salt of iminodisuccinic acid.

18. The product according to claim 15, wherein the iminodisuccinic acid or salt thereof is present in an amount from 0.001 to 15% by weight, based on the total weight of the formulation.

19. The product according to claim 15, wherein the iminodisuccinic acid or salt thereof is present in an amount from 0.1 to 0.5% by weight, based on the total weight of the formulation.

20. The product according to claim 15, wherein the cosmetic or dermatological product is a skin care product.

21. The product according to claim 15, wherein the cosmetic or dermatological product is a face care product.

22. The product according to claim 15, wherein the cosmetic or dermatological product is a sunscreen product.
